(19) United States
(12) Patent Application Publication
(10) Pub. No.: US 2024/0138779 A1
(43) Pub. Date: May 2, 2024

(54) PHYSIOLOGICAL INFORMATION PROCESSING APPARATUS AND PHYSIOLOGICAL INFORMATION PROCESSING METHOD

(71) Applicant: NIHON KOHDEN CORPORATION, Tokyo (JP)

(72) Inventors: Tetsuri ARIYAMA, Tokorozawa-shi (JP); Kenji OHARA, Tokorozawa-shi (JP); Minoru MATSUSHIMA, Tokorozawa-shi (JP); Hideki OCHIAI, Tokorozawa-shi (JP)

(73) Assignee: NIHON KOHDEN CORPORATION, Tokyo (JP)

(21) Appl. No.: 18/487,415

(22) Filed: Oct. 16, 2023

(30) Foreign Application Priority Data
Oct. 26, 2022 (JP) 2022-171640

(51) Int. Cl. A61B 5/00; A61B 5/0205

(52) U.S. Cl. CPC A61B 5/7285; A61B 5/0205; A61B 5/7221; A61B 5/746; A61B 5/02438

(57) ABSTRACT

A physiological information processing apparatus includes one or more processors, and one or more memories configured to store a computer readable instruction, when the computer readable instruction is executed by the processor, the physiological information processing apparatus is configured to: cause a plurality of physiological sensors to intermittently operate such that an operation time and a standby time are alternately repeated; obtain a plurality of pieces of physiological information on a patient from the plurality of physiological sensors during the operation time; and change an operation mode of the plurality of physiological sensors that intermittently operate in accordance with a condition related to at least a part of the plurality of pieces of physiological information or a condition related to at least a part of the plurality of physiological sensors.

### CROSS-REFERENCE TO RELATED APPLICATIONS

This application is based on and claims priority under 35 USC 119 from Japanese Patent Application No. 2022-171640 filed on Oct. 26, 2022, the contents of which are incorporated herein by reference.

### TECHNICAL FIELD

The presently disclosed subject matter relates to a physiological information processing apparatus and a physiological information processing method. The presently disclosed subject matter further relates to a program for causing a computer to execute the physiological information processing method.

### BACKGROUND ART

JP 2016-140642A discloses a physiological information measuring apparatus that can be attached to a patient and can measure physiological information such as pulse waves of the patient. JP 2016-140642A discloses a technique of, in order to reduce power consumption of the physiological information measuring apparatus attached to the patient, switching an operation mode of a physiological sensor mounted on the measuring apparatus from a continuous operation mode to an intermittent operation mode (a discontinuous operation mode) in accordance with a use condition of the measuring apparatus or the physiological information. Here, the intermittent operation mode of the physiological sensor is an operation mode in which an operation time and a standby time of the physiological sensor are alternately repeated.

However, when a normal operation mode of the physiological sensor is the continuous operation mode, the power consumption of a battery built into the measuring apparatus drastically increases, and thus it is necessary to charge the measuring apparatus every time when a specified period of time has passed. Therefore, the physiological information on the patient cannot be monitored at all times by the measuring apparatus. As described above, from the viewpoint of restraining the power consumption of the built-in battery type measuring apparatus, it is preferable that the operation mode of the physiological sensor mounted on the measuring apparatus is set to the intermittent operation mode (the discontinuous operation mode) at all times.

However, when the operation mode of the physiological sensor is set to the intermittent operation mode at all times, it is also assumed that the physiological information on the patient cannot be accurately measured during one cycle of the intermittent operation mode due to movements of the patient such as walking. In such a situation, it is desirable to prevent a decrease in measurement accuracy relating to the physiological information on the patient. Further, when symptoms of the patient are serious, it is desirable that the physiological information on the seriously ill patient can be frequently measured by increasing an operation frequency of the physiological sensor. Accordingly, there is still room to examine a physiological information processing apparatus capable of optimizing an intermittent operation of a physiological sensor according to conditions of a patient.

### SUMMARY

An object of the presently disclosed subject matter is to provide a physiological information processing apparatus and a physiological information processing method that are capable of optimizing an intermittent operation of physiological sensors according to conditions of a patient. Another object of the presently disclosed subject matter is to provide a program for causing a computer to execute the physiological information processing method.

A physiological information processing apparatus according to a first aspect of the presently disclosed subject matter includes one or more processors and one or more memories that store a computer readable instruction. When the computer readable instruction is executed by the processor, the physiological information processing apparatus is configured to: cause a plurality of physiological sensors to intermittently operate such that an operation time and a standby time are alternately repeated; obtain a plurality of pieces of physiological information on a patient from the plurality of physiological sensors during the operation time; and change an operation mode of the plurality of physiological sensors that intermittently operate in accordance with a condition related to at least a part of the plurality of pieces of physiological information or a condition related to at least a part of the plurality of physiological sensors.

According to the above configuration, the operation mode of the plurality of physiological sensors that intermittently operate (for example, remeasurement of the physiological information, extension or reduction of the operation time or the standby time) is changed in accordance with the condition related to at least a part of the plurality of pieces of physiological information or the condition related to at least a part of the plurality of physiological sensors. As described above, for example, even in a situation where the physiological information of the physiological sensors cannot be temporarily accurately measured due to movements such as motions (for example, walking) of the patient, it is possible to suitably prevent a decrease in measurement accuracy of the physiological information measured by the physiological sensors while restraining power consumption of the physiological information processing apparatus. Further, when symptoms of the patient are serious, it is possible to obtain more physiological information on the seriously ill patient from the physiological sensors while restraining the power consumption of the physiological information processing apparatus. Accordingly, it is possible to provide a physiological information processing apparatus that is capable of optimizing an intermittent operation of physiological sensors according to conditions of a patient.

A physiological information processing apparatus according to a second aspect of the presently disclosed subject matter includes one or more processors and one or more memories that store a computer readable instruction. When the computer readable instruction is executed by the processor, the physiological information processing apparatus is configured to: cause a plurality of physiological sensors to intermittently operate such that an operation time and a standby time are alternately repeated; obtain a plurality of pieces of physiological information on a patient from the plurality of physiological sensors during the operation time; determine whether a first physiological sensor among the plurality of physiological sensors is in contact with the skin of the patient based on first physiological information obtained by the first physiological sensor; and present an alarm to the patient when the first physiological sensor is not in contact with the skin of the patient.

According to the above configuration, when the first physiological sensor is not in contact with the skin of the patient, the alarm is presented to the patient. In this manner, the patient can immediately recognize that the physiological information on the patient is not accurately measured by the physiological information processing apparatus by the alarm. As described above, it is possible to suitably prevent a situation where the physiological information on the patient cannot be measured for a long period by the physiological sensors.

A physiological information processing method according to a third aspect of the presently disclosed subject matter is executed by a computer, and the physiological information processing method includes: causing a plurality of physiological sensors to intermittently operate such that an operation time and a standby time are alternately repeated; obtaining a plurality of pieces of physiological information on a patient from the plurality of physiological sensors during the operation time; and changing an operation mode of the plurality of physiological sensors that intermittently operate in accordance with a condition related to at least a part of the plurality of pieces of physiological information or a condition related to at least a part of the plurality of physiological sensors.

According to the above method, it is possible to provide a physiological information processing method that is capable of optimizing an intermittent operation of physiological sensors according to conditions of a patient.

A physiological information processing method according to a fourth aspect of the presently disclosed subject matter is executed by a computer, and the physiological information processing method includes: causing a plurality of physiological sensors to intermittently operate such that an operation time and a standby time are alternately repeated; obtaining a plurality of pieces of physiological information on a patient from the plurality of physiological sensors during the operation time; determining whether a first physiological sensor among the plurality of physiological sensors is in contact with the skin of the patient based on first physiological information obtained by the first physiological sensor; and presenting an alarm to the patient when the first physiological sensor is not in contact with the skin of the patient.

According to the above method, it is possible to suitably prevent the situation where the physiological information on the patient cannot be measured for a long period by the physiological sensors.

In addition, a program for causing the computer to execute the physiological information processing method and a non-transitory computer readable storage medium storing the program are provided.

According to the presently disclosed subject matter, it is possible to provide a physiological information processing apparatus and a physiological information processing method that are capable of optimizing an intermittent operation of physiological sensors according to conditions of a patient.

### BRIEF DESCRIPTION OF DRAWINGS

FIG. 1 is a schematic diagram illustrating an example of a physiological information processing system according to an embodiment of the presently disclosed subject matter.

FIG. 2 illustrates an example of a hardware configuration of a physiological information processing apparatus according to an embodiment of the presently disclosed subject matter.

FIG. 3 is a flow chart for explaining a basic intermittent operation of physiological sensors.

FIG. 4 is a time chart illustrating the basic intermittent operation of the physiological sensors.

FIG. 5 is a flow chart for explaining an intermittent operation of the physiological sensors according to a first embodiment.

FIG. 6 is a time chart illustrating an example of the intermittent operation of the physiological sensors according to the first embodiment.

FIG. 7 is a flow chart for explaining an intermittent operation of the physiological sensors according to a first modification of the first embodiment.

FIG. 8 is a flow chart for explaining an intermittent operation of the physiological sensors according to a second modification of the first embodiment.

FIG. 9 is a flow chart for explaining an intermittent operation of the physiological sensors according to a second embodiment.

FIG. 10 is a time chart illustrating an example of the intermittent operation of the physiological sensors according to the second embodiment.

FIG. 11 is a flow chart for explaining an intermittent operation of the physiological sensors according to a third embodiment.

FIG. 12 is a table for explaining an example of a method for calculating a NEWS score.

FIG. 13 illustrates an example of a temporal transition of a calculated symptom severity score.

FIG. 14 is a time chart illustrating an example of the intermittent operation of the physiological sensors according to the third embodiment.

FIG. 15 is a flow chart for explaining an intermittent operation of the physiological sensors according to a fourth embodiment.

FIG. 16 is a time chart illustrating an example of the intermittent operation of the physiological sensors according to the fourth embodiment.

FIG. 17 is a flow chart for explaining a process of presenting an alarm to a patient.

FIG. 18 is a time chart illustrating an example of the intermittent operation of the physiological sensors, which includes an alarm presentation time.

### DESCRIPTION OF EMBODIMENTS

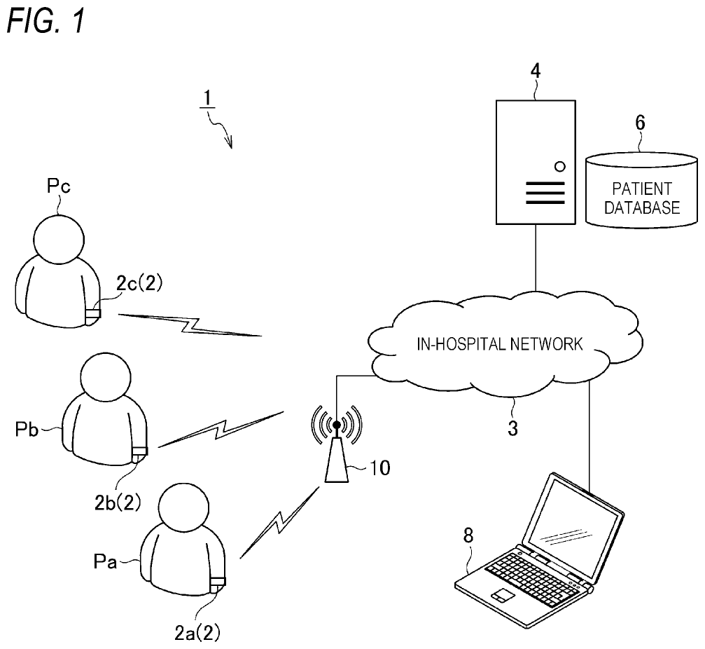

First, a physiological information processing system 1 (hereinafter, simply referred to as the “processing system 1”) according to an embodiment of the presently disclosed subject matter (hereinafter, referred to as the present embodiment) will be described with reference to FIG. 1. FIG. 1 is a schematic diagram illustrating an example of the processing system 1 according to the present embodiment.

As illustrated in FIG. 1, the processing system 1 is a communication system constructed in a hospital, and can include a plurality of physiological information processing apparatuses 2a to 2c, a server 4, and an information terminal 8. The physiological information processing apparatuses 2a to 2c, the server 4, and the information terminal 8 are connected to an in-hospital network 3. The in-hospital network 3 is constructed by, for example, a local area network (LAN) or a wide area network (WAN). The physiological information processing apparatuses 2a to 2c are respectively attached to patients Pa to Pc in the hospital. In the following description, the physiological information processing apparatuses 2a to 2c are simply referred to as the processing apparatuses 2a to 2c. Further, for the sake of convenient description, the processing apparatuses 2a to 2c may be collectively referred to as the processing apparatus 2, and the patients Pa to Pc may be simply referred to as the patient P.

The processing apparatus 2 is a wearable medical device to be attached to a part of the body of the patient P (a subject), and obtains physiological information data of the patient P. The processing apparatus 2 has a wireless communication function and is communicably connected to the in-hospital network 3 via a wireless access point 10 installed in the hospital. The processing apparatus 2 can obtain the physiological information data of the patient P and then transmit the physiological information data of the patient P to the server 4 via the wireless access point 10 and the in-hospital network 3.

The server 4 stores the physiological information data of the patient P transmitted from the processing apparatus 2 in a patient database 6. The patient database 6 stores the physiological information data and attribute information of the patient P. The information terminal 8 can access the server 4 via the in-hospital network 3. The information terminal 8 can obtain information related to the physiological information data of the patient P from the server 4 and then display the obtained information on a display.

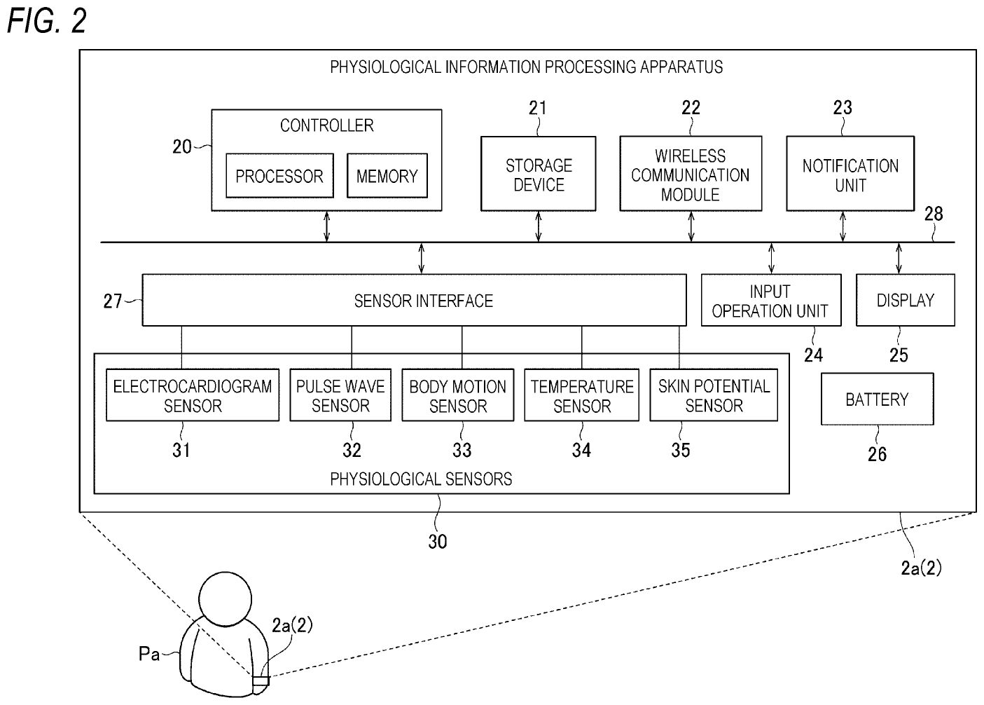

Next, a hardware configuration of the processing apparatus 2a will be described with reference to FIG. 2. FIG. 2 illustrates an example of the hardware configuration of the processing apparatus 2a according to the present embodiment. In the present embodiment, the processing apparatuses 2a to 2c have the same configuration. Hereinafter, the processing apparatus 2a is simply referred to as the processing apparatus 2, and the patient Pa is simply referred to as the patient P.

As illustrated in FIG. 2, the processing apparatus 2 can include a controller 20, a storage device 21, a wireless communication module 22, a notification unit 23, a display 25, an input operation unit 24, and a sensor interface 27. These components are communicably connected to each other via a bus 28.

The controller 20 can include one or more memories and one or more processors. The memory stores a computer readable instruction (a program). For example, the memory may include a read only memory (ROM) in which various programs are stored, a random access memory (RAM) having a plurality of work areas in which various programs to be executed by the processor are stored, and the like. The processor may include at least one of, for example, a central processing unit (CPU), a micro processing unit (MPU), and a graphics processing unit (GPU). The CPU may include a plurality of CPU cores. The GPU may include a plurality of GPU cores. The processor may load a program designated from various programs incorporated in the storage device 21 or the ROM in the RAM, and execute various processes in cooperation with the RAM. Since the processor loads a physiological information processing program to be described later in the RAM and executes the program in cooperation with the RAM, the controller 20 may control various operations of the processing apparatus 2. Details of the physiological information processing program will be described later.

The storage device 21 is, for example, a storage device such as a flash memory, and stores programs and various data. The physiological information processing program may be incorporated in the storage device 21. Further, the physiological information data of the patient P such as electrocardiogram data, pulse wave data, body motion data, temperature data, and skin potential data may be stored in the storage device 21.

The wireless communication module 22 connects the processing apparatus 2 to the in-hospital network 3. The wireless communication module 22 may include an RF circuit for performing wireless communication with the wireless access point 10, and a transmission and reception antenna. A short-distance wireless communication standard between the wireless access point 10 and the processing apparatus 2 is, for example, Wi-Fi (registered trademark) or Bluetooth (registered trademark).

The notification unit 23 presents an alarm (a warning) to the patient P. For example, the notification unit 23 visually presents the alarm to the patient P, and may include a light emitting indicator having at least one light emitter (for example, an LED). The notification unit 23 may include a voice output unit (a speaker) that audibly presents an alarm to the patient P. Further, the notification unit 23 may include a vibration generator that tactically presents an alarm to the patient P.

The display 25 displays information related to the physiological information on the patient P, and may include, for example, a liquid crystal panel, an organic EL panel, or electronic paper. The input operation unit 24 receives an input operation by the patient P and generates an instruction signal corresponding to the input operation. The input operation unit 24 is, for example, a touch panel disposed on the display 25 in an overlapping manner, an operation button installed on a housing of the processing apparatus 2. After the instruction signal generated by the input operation unit 24 is transmitted to the controller 20 via the bus 28, the controller 20 executes a predetermined operation according to the instruction signal.

The sensor interface 27 is an interface for communicably connecting an electrocardiogram sensor 31, a pulse wave sensor 32, a body motion sensor 33, a temperature sensor 34, and a skin potential sensor 35 to the processing apparatus 2. In the following description, the electrocardiogram sensor 31, the pulse wave sensor 32, the body motion sensor 33, the temperature sensor 34, and the skin potential sensor 35 may be collectively referred to as physiological sensors 30. The sensor interface 27 may include a plurality of input terminals through which physiological signals output from the plurality of physiological sensors 30 are input. Each input terminal may be physically connected to a connector of the corresponding physiological sensor 30. Further, the sensor interface 27 may include a wireless communication circuit for wirelessly communicating with the plurality of physiological sensors 30, an antenna, and the like. The sensor interface 27 may include an analog processing circuit (for example, a filter processing circuit, a signal amplification circuit, an AD converter, or the like) for processing the signals output from the physiological sensors 30. In this manner, analog physiological signals output from the physiological sensors 30 may be converted into digital physiological signals by the sensor interface 27.

The processing apparatus 2 can further include the electrocardiogram sensor 31, the pulse wave sensor 32, the body motion sensor 33, the temperature sensor 34, and the skin potential sensor 35 (that is, the plurality of physiological sensors 30). The electrocardiogram sensor 31 is attached to the chest and/or a hand and a feet of the patient P, and obtains an electrocardiogram signal indicating a temporal change in an action potential of the heart of the patient P. The pulse wave sensor 32 (for example, a SpO2 sensor) is attached to a fingertip or a wrist of the patient P, and obtains a pulse wave signal indicating a temporal change in a pulse wave of the patient P. The body motion sensor 33 is, for example, an acceleration sensor, and obtains a body motion signal indicating a temporal change in a body motion of the patient P. The temperature sensor 34 is in contact with the skin of the patient P, and obtains a temperature signal indicating a temporal change in the temperature of the patient P. The skin potential sensor is in contact with the skin of the patient P, and obtains a skin potential signal indicating a temporal change in a skin potential of the patient P.

In this manner, the controller 20 may obtain the electrocardiogram data indicating an electrocardiogram waveform of the patient P based on the electrocardiogram signal from the electrocardiogram sensor 31, and may obtain heart rate data indicating a temporal change in a heart rate of the patient P based on the electrocardiogram data. Further, the controller 20 may obtain respiration rate data indicating a temporal change in a respiration rate of the patient P based on the electrocardiogram signal. The controller 20 may obtain the pulse wave data indicating a temporal change in the pulse wave of the patient P based on the pulse wave signal from the pulse wave sensor 32, and may also obtain pulse rate data indicating a temporal change in a pulse rate of the patient P and SpO2 data indicating a temporal change in a transcutaneous arterial oxygen saturation (SpO2) of the patient P based on the pulse wave data.

Same or similarly, the controller 20 may obtain the body motion data (acceleration data) indicating the temporal change in the body motion (acceleration) of the patient P from the body motion sensor 33. The controller 20 may obtain the temperature data indicating the temporal change in the temperature of the patient P from the temperature sensor 34, and may obtain the skin potential data indicating the temporal change in the skin potential of the patient P from the skin potential sensor 35. The electrocardiogram data, the heart rate data, the pulse wave data, the pulse rate data, the SpO2 data, the body motion data, the temperature data, and the skin potential data are examples of the physiological information on the patient P.

In the example of the processing apparatus 2 illustrated in FIG. 2, the five physiological sensors 30 are provided in the processing apparatus 2, but at least one of the electrocardiogram sensor 31, the pulse wave sensor 32, the body motion sensor 33, the temperature sensor 34, and the skin potential sensor 35 may be provided in the processing apparatus 2. For example, in a first embodiment to be described later, at least one of the electrocardiogram sensor 31, the pulse wave sensor 32, and the body motion sensor 33 may be provided in the processing apparatus 2.

Further, the processing apparatus 2 is provided with a battery 26. The components of the processing apparatus 2 operate by power supplied from the battery 26. In the present embodiment, in order to restrain consumption of the power supplied from the battery 26, the controller 20 causes the physiological sensors 30 to intermittently operate such that an operation time and a standby time are alternately repeated. In other words, the controller 20 intermittently measures the physiological information on the patient P by using the physiological sensors 30 such that a measurement time and the standby time are alternately repeated. Here, the operation time of the physiological sensors 30 is a time during which the physiological information is obtained by using the physiological sensors 30. That is, the operation time of the physiological sensors 30 corresponds to the measurement time during which the physiological information is measured by using the physiological sensors 30. On the other hand, the standby time of the physiological sensors 30 is a time during which the physiological information is not measured by using the physiological sensors 30. For example, an operation time of the pulse wave sensor 32 corresponds to a measurement time during which the pulse wave data is measured by using the pulse wave sensor 32. On the other hand, a standby time of the pulse wave sensor 32 corresponds to a time during which the pulse wave data is not measured by using the pulse wave sensor 32.

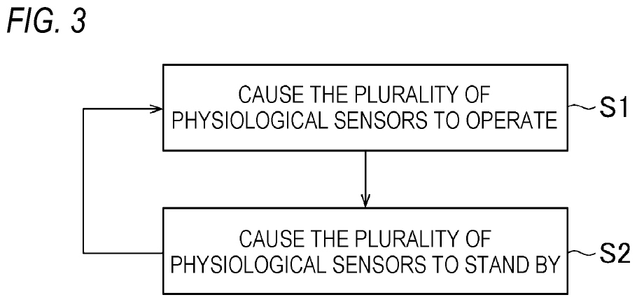

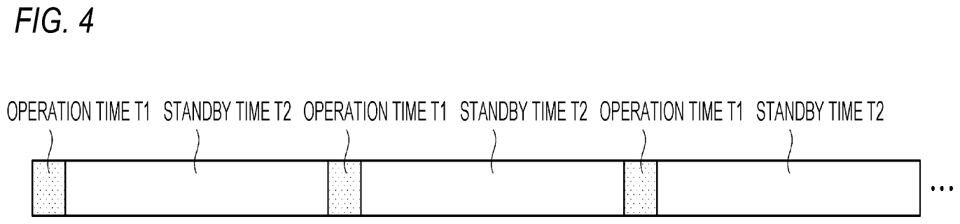

Next, a basic intermittent operation of the physiological sensors 30 will be described with reference to FIGS. 3 and 4. FIG. 3 is a flow chart for explaining the basic intermittent operation of the physiological sensors 30. As illustrated in FIG. 3, the controller 20 causes the plurality of physiological sensors 30 to operate in step S1. That is, the controller 20 measures a plurality of pieces of physiological information by using the plurality of physiological sensors 30. Thereafter, the controller 20 causes the plurality of physiological sensors 30 to stand by in step S2. That is, the controller 20 does not measure the plurality of pieces of physiological information. Then, the process in step S1 and the process in step S2 are repeatedly executed. As illustrated in FIG. 4, in a time chart illustrating the basic intermittent operation of the physiological sensors 30, an operation time T1 in step S1 and a standby time T2 in step S2 are alternately repeated. In this example, from the viewpoint of reducing power consumption, the operation time T1 is shorter than the standby time T2. The operation time T1 is, for example, 1 minute. The standby time T2 is, for example, 14 minutes.

On the other hand, in a case where the physiological sensors 30 operate intermittently so that the operation time T1 and the standby time T2 are alternately repeated as described above, a situation where the physiological information on the patient P cannot be accurately measured in one cycle including the operation time T1 and the standby time T2 is also assumed. For example, when the patient P performs exercise such as walking during the operation time T1, there is a possibility that a measurement accuracy of the physiological information decreases due to a body movement of the patient P. When symptoms of the patient P become serious, it is desirable that a measurement frequency of the physiological information on the patient P can be increased by increasing a percentage of the operation time T1 in one cycle.

In view of such a situation, in each embodiment of the intermittent operation of the physiological sensors 30 to be described below, a current situation of the patient P is estimated by using the physiological information, and then the intermittent operation of the physiological sensors 30 is optimized according to the estimated current situation of the patient P.

### First Embodiment

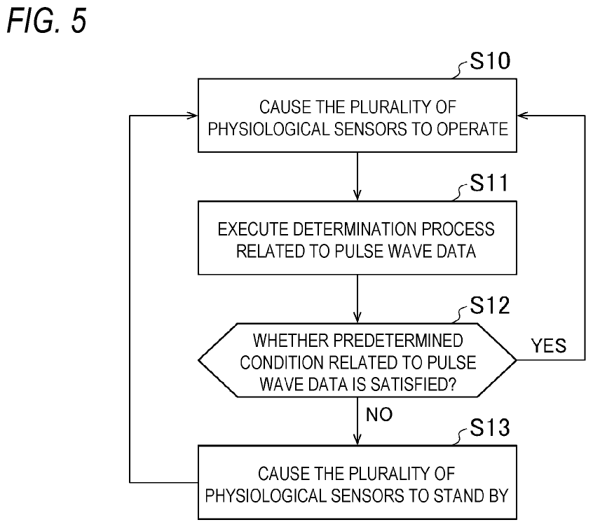

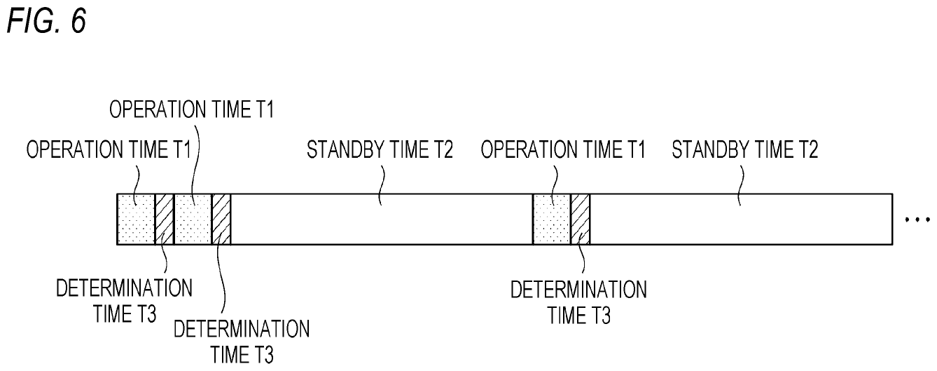

First, the intermittent operation of the physiological sensors 30 according to the first embodiment will be described below with reference to FIGS. 5 and 6. FIG. 5 is a flow chart for explaining the intermittent operation of the physiological sensors 30 according to the first embodiment. FIG. 6 is a time chart illustrating an example of the intermittent operation of the physiological sensors 30 according to the first embodiment.

As illustrated in FIG. 5, in step S10, the controller 20 causes the physiological sensors (the electrocardiogram sensor 31, the pulse wave sensor 32, the body motion sensor 33, the temperature sensor 34, and the skin potential sensor 35) to operate during the operation time T1 (see FIG. 6) so as to obtain the plurality of pieces of physiological information (the electrocardiogram data, the heart rate data, the pulse wave data, the pulse rate data, the SpO2 data, the body motion data, the temperature data, and the skin potential data). In step S11, the controller 20 executes a determination process related to the pulse wave data during a determination time T3. A specific example of the determination process in step S11 will be described later. In the time chart illustrated in FIG. 6, the operation time T1 and the determination time T3 do not temporally overlap, but the operation time T1 and the determination time T3 may at least partially overlap.

Next, when the controller 20 determines that the pulse wave data does not satisfy a predetermined condition related to the pulse wave data (NO in step S12) as a result of the determination process in step S11, the controller 20 causes the physiological sensors 30 to stand by during the standby time T2 (step S13). Thereafter, the controller 20 obtains the physiological information by causing the physiological sensors 30 to operate again (step S10).

On the other hand, when the controller 20 determines that the pulse wave data satisfies the predetermined condition related to the pulse wave data (YES in step S12) as the result of the determination process in step S11, the controller 20 obtains the physiological information by causing the physiological sensors 30 to operate again without causing the physiological sensors 30 to stand by during the standby time T2 (step S10).

As described above, due to the intermittent operation of the physiological sensors 30 according to the first embodiment, when the predetermined condition related to the pulse wave data is satisfied, the physiological sensors 30 operate again without standing by. On the other hand, when the predetermined condition related to the pulse wave data is not satisfied, the physiological sensors 30 operate again after standing by during the standby time T2.

In the example of the time chart illustrated in FIG. 6, since the determination result of the first step S12 is YES, the physiological sensors 30 operate again without standing by. Thereafter, since the determination result of the next step S12 is NO, the physiological sensors operate again after standing by during the standby time T2.

Specific Example of Determination Process Related to Pulse Wave Data

Next, the determination process related to the pulse wave data in step S11 will be specifically described below. In this regard, it is determined whether pulse waves included in the pulse wave data are irregular in the determination process in step S11. When the pulse waves are irregular (YES in step S12), it is estimated that the measurement accuracy of the physiological information decreases due to the motions of the patient, and thus the controller causes the physiological sensors 30 to operate again without standing by. As described above, in a case where it is estimated that the measurement accuracy of the physiological information decreases, it is possible to compensate for the decrease in the measurement accuracy of the physiological information by increasing the measurement frequency of the physiological information.

Determination Process Based on Comparison Between Each Pulse Wave and Reference Waveform

In step S11, the controller 20 may determine whether the pulse waves included in the pulse wave data are irregular by comparing each pulse wave included in the pulse wave data with a reference waveform (a standard waveform). For example, the controller 20 may calculate a coincidence degree (a percentage) between each pulse wave included in the pulse wave data and the reference waveform, and then compare a representative value (for example, an average value, a median value, a maximum value, or a minimum value) of the coincidence degree of each pulse wave with a predetermined threshold value. When the representative value of the coincidence degree of each pulse wave is smaller than the predetermined threshold value, it may be determined that the pulse waves are irregular. That is, it may be determined that the pulse wave data satisfies the predetermined condition. On the other hand, when the representative value of the coincidence degree of each pulse wave is equal to or larger than the predetermined threshold value, it may be determined that the pulse waves are not irregular. That is, it may be determined that the pulse wave data does not satisfy the predetermined condition. Here, the reference waveform may be generated by synthesizing a plurality of pulse waves included in past pulse wave data of the patient P, or may be a standard pulse wave waveform determined according to an attribute of the patient P. Further, the coincidence degree (a similarity) between each pulse wave and the reference waveform may be determined based on at least one of a height, a width, a shape, and other feature quantities of the pulse wave.

Determination Process Based on Frequency Characteristics of Pulse Wave Data

The controller 20 may determine whether the pulse waves included in the pulse wave data are irregular by analyzing frequency characteristics of the pulse wave data. For example, the controller 20 may determine whether a peak of a waveform spectrum exists within a predetermined frequency range by performing frequency analysis on a series of continuously appearing pulse waves that are indicated by the pulse wave data. The predetermined frequency range is, for example, a range of 0 Hz to 5 Hz. When the peak of the waveform spectrum exists within the predetermined frequency range, it may be determined that the pulse waves are irregular. That is, it may be determined that the pulse wave data satisfies the predetermined condition. On the other hand, when no waveform spectrum exists within the predetermined frequency range, it may be determined that the pulse waves are not irregular. That is, it may be determined that the pulse wave data does not satisfy the predetermined condition. Further, the controller 20 may determine whether N or more (N is an integer of 2 or more) peaks of the waveform spectrum exist within the predetermined frequency range. In this case, when the N or more peaks of the waveform spectrum exist within the predetermined frequency range, the controller 20 may determine that the pulse waves are irregular. On the other hand, when the N or more peaks of the waveform spectrum do not exist within the predetermined frequency range, the controller 20 may determine that the pulse waves are not irregular.

Determination Process Based on Height of Each Pulse Wave

The controller 20 may determine whether the pulse waves are irregular based on the heights of the pulse waves included in the pulse wave data. Specifically, the controller 20 may determine whether the height of each pulse wave is larger than a predetermined threshold value, and then determine that the pulse waves are irregular when the number of pulse waves higher than the predetermined threshold value is several or more. On the other hand, when the number of pulse waves higher than the predetermined threshold value is less than several, it may be determined that the pulse waves are not irregular.

Determination Process Based on Presence or Absence of Arrhythmia

The controller 20 may determine whether arrhythmia exists in the pulse waves included in the pulse wave data. For example, the controller 20 may determine whether each pulse wave corresponds to one of a plurality of types of arrhythmia waveforms based on parameters of the pulse wave (for example, the height, the width, and the shape of the pulse wave) included in the pulse wave data. When at least one of the plurality of pulse waves included in the pulse wave data corresponds to one of the plurality of types of arrhythmia waveforms, it may be determined that the pulse waves are irregular. On the other hand, when none of the pulse waves corresponds to the arrhythmia waveforms, it may be determined that the pulse waves are not irregular.

As described above, by the intermittent operation of the physiological sensors 30 according to the first embodiment, when the pulse wave data does not satisfy the predetermined condition (that is, when the pulse waves are not irregular), the plurality of physiological sensors operate after standing by during the standby time. On the other hand, when the pulse wave data satisfies the predetermined condition (that is, when the pulse waves are irregular), the plurality of physiological sensors 30 operate again without standing by. As described above, in a situation where there is a risk that the physiological information of the physiological sensors cannot be temporarily accurately measured due to movements such as motions of the patient P, the physiological sensors 30 operate again without standing by. Therefore, it is possible to suitably prevent the decrease in the measurement accuracy of the physiological information measured by the physiological sensors 30 while restraining the power consumption of the processing apparatus 2.

### First Modification of First Embodiment

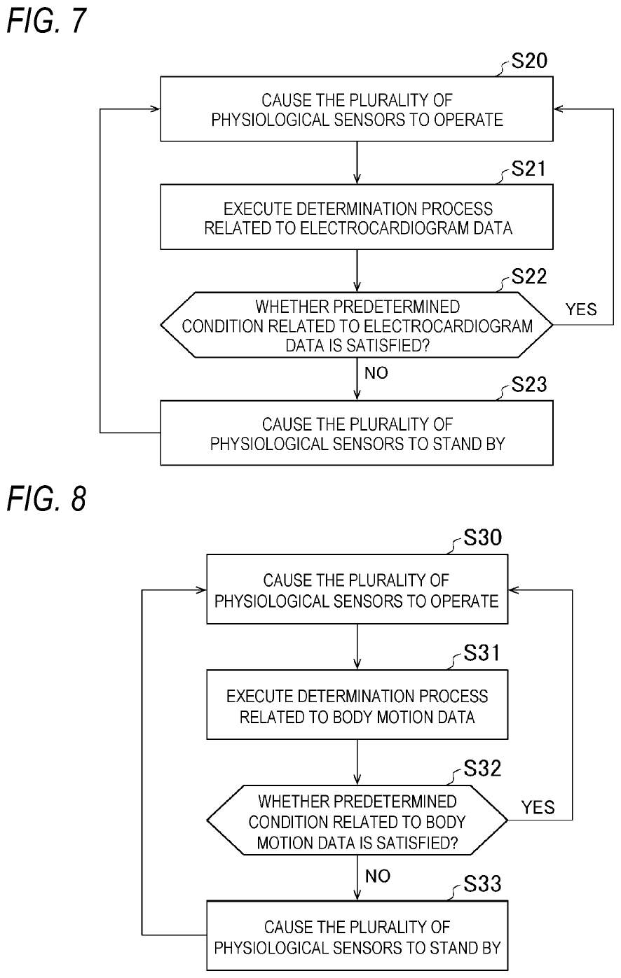

Next, an intermittent operation of the physiological sensors 30 according to a first modification of the first embodiment will be described below with reference to FIG. 7. FIG. 7 is a flow chart for explaining the intermittent operation of the physiological sensors 30 according to the first modification of the first embodiment. As illustrated in FIG. 7, in step S20, the controller 20 causes the physiological sensors 30 (the electrocardiogram sensor 31, the pulse wave sensor 32, the body motion sensor 33, the temperature sensor 34, and the skin potential sensor 35) to operate during the operation time T1 (see FIG. 6) so as to obtain the plurality of pieces of physiological information (the electrocardiogram data, the heart rate data, the pulse wave data, the pulse rate data, the SpO2 data, the body motion data, the temperature data, and the skin potential data). In step S21, the controller 20 executes a determination process related to the electrocardiogram data during the determination time T3. A specific example of the determination process in step S21 will be described later.

Next, when the controller 20 determines that the electrocardiogram data does not satisfy a predetermined condition related to the electrocardiogram data (NO in step S22) as a result of the determination process in step S21, the controller 20 causes the physiological sensors 30 to stand by during the standby time T2 (step S23). Thereafter, the controller 20 obtains the physiological information by causing the physiological sensors 30 to operate again (step S20).

On the other hand, when the controller 20 determines that the electrocardiogram data satisfies the predetermined condition related to the electrocardiogram data (YES in step S22) as the result of the determination process in step S21, the controller 20 obtains the physiological information by causing the physiological sensors 30 to operate again without causing the physiological sensors 30 to stand by during the standby time T2 (step S20).

As described above, due to the intermittent operation of the physiological sensors 30 according to the first modification of the first embodiment, when the predetermined condition related to the electrocardiogram data is satisfied, the physiological sensors 30 operate again without standing by. On the other hand, when the predetermined condition related to the electrocardiogram data is not satisfied, the physiological sensors 30 operate again after standing by during the standby time T2.

Specific Example of Determination Process Related to Electrocardiogram Data

Next, the determination process related to the electrocardiogram data in step S21 will be specifically described below. In this regard, it is determined whether heart rate waveforms included in the electrocardiogram data are irregular in the determination process in step S21. When the heart rate waveforms are irregular (YES in step S22), it is estimated that the measurement accuracy of the physiological information decreases due to the motions of the patient, and thus the controller 20 causes the physiological sensors 30 to operate again without standing by. As described above, in the case where it is estimated that the measurement accuracy of the physiological information decreases, it is possible to compensate for the decrease in the measurement accuracy of the physiological information by increasing the measurement frequency of the physiological information.

Determination Process Based on Comparison Between Each Heart Rate Waveform and Reference Waveform

In step S21, the controller 20 may determine whether the heart rate waveforms included in the electrocardiogram data are irregular by comparing each heart rate waveform included in the electrocardiogram data with a reference waveform (a standard waveform). For example, the controller 20 may calculate a coincidence degree (a percentage) between each heart rate waveform included in the electrocardiogram data and the reference waveform, and then compare a representative value (for example, an average value, a median value, a maximum value, or a minimum value) of the coincidence degree of each heart rate waveform with a predetermined threshold value. When the representative value of the coincidence degree of each heart rate waveform is smaller than the predetermined threshold value, it may be determined that the heart rate waveforms are irregular. That is, it may be determined that the electrocardiogram data satisfies the predetermined condition. On the other hand, when the representative value of the coincidence degree of each heart rate waveform is equal to or larger than the predetermined threshold value, it may be determined that the heart rate waveforms are not irregular. That is, it may be determined that the electrocardiogram data does not satisfy the predetermined condition. Here, the reference waveform may be generated by synthesizing a plurality of heart rate waveforms included in past electrocardiogram data of the patient P, or may be a standard heart rate waveform determined according to an attribute of the patient P. Further, the coincidence degree (the similarity) between each heart rate waveform and the reference waveform may be determined based on at least one of a height, a width, a shape, and other feature quantities of the heart rate waveform.

Determination Process Based on Frequency Characteristics of Electrocardiogram Data

The controller 20 may determine whether the heart rate waveforms included in the electrocardiogram data are irregular by analyzing frequency characteristics of the electrocardiogram data. For example, the controller 20 may determine whether a peak of a waveform spectrum exists within a predetermined frequency range (for example, a range of 1.5 Hz to 2.5 Hz) by performing frequency analysis on a series of continuously appearing heart rate waveforms that are indicated by the electrocardiogram data. When the peak of the waveform spectrum exists within the predetermined frequency range, it may be determined that the heart rate waveforms are irregular. That is, it may be determined that the electrocardiogram data satisfies the predetermined condition. On the other hand, when no waveform spectrum exists within the predetermined frequency range, it may be determined that the heart rate waveforms are not irregular. That is, it may be determined that the electrocardiogram data does not satisfy the predetermined condition.

Determination Process Based on Height of Each Heart Rate Waveform

The controller 20 may determine whether the heart rate waveforms are irregular based on the heights of the heart rate waveforms included in the electrocardiogram data. Specifically, the controller 20 may determine whether the height of each heart rate waveform is larger than a predetermined threshold value, and then determine that the heart rate waveforms are irregular when the number of heart rate waveforms higher than the predetermined threshold value is several or more. On the other hand, when the number of heart rate waveforms higher than the predetermined threshold value is less than several, it may be determined that the heart rate waveforms are not irregular.

Determination Process Based on Presence or Absence of Arrhythmia

The controller 20 may determine whether arrhythmia exists in the heart rate waveforms included in the electrocardiogram data. For example, the controller 20 may determine whether each heart rate waveform corresponds to one of the plurality of types of arrhythmia waveforms based on parameters of the heart rate waveform (for example, the height, the width, and the shape of the heart rate waveform) included in the electrocardiogram data. When at least one of the plurality of heart rate waveforms included in the electrocardiogram data corresponds to one of the plurality of types of arrhythmia waveforms, it may be determined that the heart rate waveforms are irregular. On the other hand, when none of the heart rate waveforms corresponds to the arrhythmia waveforms, it may be determined that the heart rate waveforms are not irregular.

As described above, by the intermittent operation of the physiological sensors 30 according to the first modification of the first embodiment, when the electrocardiogram data does not satisfy the predetermined condition (that is, when the heart rate waveforms are not irregular), the plurality of physiological sensors 30 operate after standing by during the standby time. On the other hand, when the electrocardiogram data satisfies the predetermined condition (that is, when the heart rate waveforms are irregular), the plurality of physiological sensors 30 operate again without standing by. As described above, in the situation where there is a risk that the physiological information of the physiological sensors 30 cannot be temporarily accurately measured due to the movements such as motions of the patient P, the physiological sensors 30 operate again without standing by. Therefore, it is possible to suitably prevent the decrease in the measurement accuracy of the physiological information measured by the physiological sensors 30 while restraining the power consumption of the processing apparatus 2.

### Second Modification of First Embodiment

Next, an intermittent operation of the physiological sensors 30 according to a second modification of the first embodiment will be described below with reference to FIG. 8. FIG. 8 is a flow chart for explaining the intermittent operation of the physiological sensors 30 according to the second modification of the first embodiment. As illustrated in FIG. 8, in step S30, the controller 20 causes the physiological sensors 30 (the electrocardiogram sensor 31, the pulse wave sensor 32, the body motion sensor 33, the temperature sensor 34, and the skin potential sensor 35) to operate during the operation time T1 (see FIG. 6) so as to obtain the plurality of pieces of physiological information (the electrocardiogram data, the heart rate data, the pulse wave data, the pulse rate data, the SpO2 data, the body motion data, the temperature data, and the skin potential data). In step S31, the controller 20 executes a determination process related to the body motion data during the determination time T3. A specific example of the determination process in step S31 will be described later.

Next, when the controller 20 determines that the body motion data does not satisfy a predetermined condition related to the body motion data (NO in step S32) as a result of the determination process in step S31, the controller 20 causes the physiological sensors 30 to stand by during the standby time T2 (step S33). Thereafter, the controller 20 obtains the physiological information by causing the physiological sensors 30 to operate again (step S30).

On the other hand, when the controller 20 determines that the body motion data satisfies the predetermined condition related to the body motion data (YES in step S32) as the result of the determination process in step S31, the controller 20 obtains the physiological information by causing the physiological sensors 30 to operate again without causing the physiological sensors 30 to stand by during the standby time T2 (step S30).

As described above, due to the intermittent operation of the physiological sensors 30 according to the second modification of the first embodiment, when the predetermined condition related to the body motion data is satisfied, the physiological sensors 30 operate again without standing by. On the other hand, when the predetermined condition related to the body motion data is not satisfied, the physiological sensors 30 operate again after standing by during the standby time T2.

Specific Example of Determination Process Related to Body Motion Data

Next, the determination process related to the body motion data (the acceleration data) in step S31 will be specifically described below. In this regard, it is determined whether the patient P moves based on the body motion data in the determination process in step S31. When the patient P moves (YES in step S32), it is estimated that the measurement accuracy of the physiological information decreases due to the motions of the patient, and thus the controller causes the physiological sensors 30 to operate again without standing by. As described above, in the case where it is estimated that the measurement accuracy of the physiological information decreases, it is possible to compensate for the decrease in the measurement accuracy of the physiological information by increasing the measurement frequency of the physiological information.

Determination Process Based on Comparison Between Average Value of Acceleration in Each Section and Predetermined Threshold Value

The controller 20 may determine whether the patient P moves based on a comparison between an average value of the acceleration in a certain section (for example, 1 second) and a predetermined threshold value. For example, when the body motion data (the acceleration data) obtained for 1 minute is divided every second, the body motion data for 1 minute is divided into 60 sections. The controller 20 calculates an average value of the acceleration in each of the 60 sections, and then compares the average value of the acceleration in each section with the predetermined threshold value. As a result of this comparison, it may be determined that the patient P moves when the number of sections in which the average value of the acceleration is equal to or larger than the predetermined threshold value exceeds half of the total (that is, when the number of sections exceeds 30). That is, it may be determined that the body motion data satisfies the predetermined condition. On the other hand, it may be determined that the patient P does not move when the number of sections in which the average value of the acceleration is equal to or larger than the predetermined threshold value is half of the total or less (that is, when the number of sections is 30 or less). That is, it may be determined that the body motion data does not satisfy the predetermined condition.

Further, in the above example, the average value of the acceleration in each section is calculated, and then the average value of the acceleration in each section is compared with the predetermined threshold value, but a variance value or an integral value of the acceleration in each section may be compared with a predetermined threshold value. In this case, it may be determined that the patient P moves when the number of sections in which the variance value or the integral value of the acceleration is equal to or larger than the predetermined threshold value exceeds half of the total (that is, when the number of sections exceeds 30). That is, it may be determined that the body motion data satisfies the predetermined condition. On the other hand, it may be determined that the patient P does not move when the number of sections in which the variance value or the integral value of the acceleration is equal to or larger than the predetermined threshold value is half of the total or less (that is, when the number of sections is 30 or less). That is, it may be determined that the body motion data does not satisfy the predetermined condition.

Determination Process Based on Comparison Between Time During which Acceleration Changes and Predetermined Threshold Value

The controller 20 may determine whether the patient P moves based on a comparison between a time during which the acceleration changes and a predetermined threshold value. In this respect, for example, the controller 20 specifies the time during which the acceleration changes in the body motion data for 1 minute, and then determines whether the time during which the acceleration changes is equal to or larger than the predetermined threshold value. When the time during which the acceleration changes is equal to or larger than the predetermined threshold value, it may be determined that the patient P moves. That is, it may be determined that the body motion data satisfies the predetermined condition. On the other hand, when the time during which the acceleration changes is smaller than the predetermined threshold value, it may be determined that the patient P does not move. That is, it may be determined that the body motion data does not satisfy the predetermined condition.

Determination Process Based on Comparison Between Time During which Value of Acceleration is not 1G and Predetermined Threshold Value

The controller 20 may determine whether the patient P moves based on a comparison between a time during which the acceleration is not 1G and a predetermined threshold value. In this respect, for example, the controller 20 specifies the time during which the acceleration is not 1G in the body motion data for 1 minute, and then determines whether the time during which the acceleration is not 1G is equal to or larger than the predetermined threshold value. When the time during which the acceleration is not 1G is equal to or larger than the predetermined threshold value, it may be determined that the patient P moves. That is, it may be determined that the body motion data satisfies the predetermined condition. On the other hand, when the time during which the acceleration is not 1G is smaller than the predetermined threshold value, it may be determined that the patient P does not move. That is, it may be determined that the body motion data does not satisfy the predetermined condition.

Determination Process Based on Frequency Characteristics of Body Motion Data

The controller 20 may determine whether the patient P moves by analyzing frequency characteristics of the body motion data. For example, the controller 20 may determine whether a peak of a waveform spectrum exists within a predetermined frequency band (for example, a frequency band of 1 Hz or more) by performing frequency analysis on a waveform indicating a temporal change in the acceleration of the patient P that is indicated by the body motion data. When the peak of the waveform spectrum exists within the predetermined frequency band, it may be determined that the patient P moves. That is, it may be determined that the body motion data satisfies the predetermined condition. On the other hand, when no waveform spectrum exists within the predetermined frequency band, it may be determined that the patient P does not move. That is, it may be determined that the body motion data does not satisfy the predetermined condition.

As described above, by the intermittent operation of the physiological sensors 30 according to the second modification of the first embodiment, when the body motion data does not satisfy the predetermined condition (that is, when the patient P does not move), the plurality of physiological sensors 30 operate after standing by during the standby time. On the other hand, when the body motion data satisfies the predetermined condition (that is, when the patient P moves), the plurality of physiological sensors 30 operate again without standing by. As described above, in the situation where there is a risk that the physiological information of the physiological sensors 30 cannot be temporarily accurately measured due to the movements such as motions of the patient P, the physiological sensors 30 operate again without standing by. Therefore, it is possible to suitably prevent the decrease in the measurement accuracy of the physiological information measured by the physiological sensors 30 while restraining the power consumption of the processing apparatus 2.

### Second Embodiment

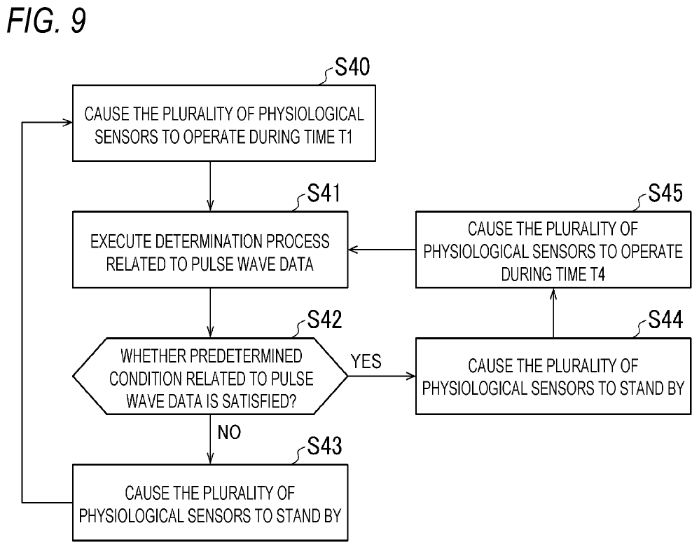

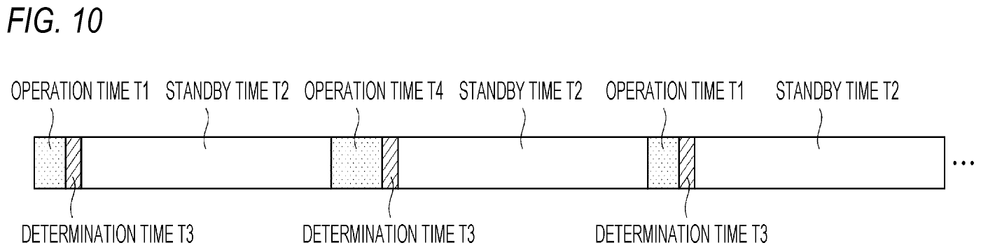

Next, an intermittent operation of the physiological sensors 30 according to a second embodiment will be described below with reference to FIGS. 9 and 10. FIG. 9 is a flow chart for explaining the intermittent operation of the physiological sensors 30 according to the second embodiment. FIG. 10 is a time chart illustrating an example of the intermittent operation of the physiological sensors 30 according to the second embodiment.

As illustrated in FIG. 9, in step S40, the controller 20 causes the physiological sensors (the electrocardiogram sensor 31, the pulse wave sensor 32, the body motion sensor 33, the temperature sensor 34, and the skin potential sensor 35) to operate during the operation time T1 (see FIG. 10) so as to obtain the plurality of pieces of physiological information (the electrocardiogram data, the heart rate data, the pulse wave data, the pulse rate data, the SpO2 data, the body motion data, the temperature data, and the skin potential data). In step S41, the controller 20 executes a determination process related to the pulse wave data during the determination time T3. A specific example of the determination process in step S41 will be described later. In the time chart illustrated in FIG. 10, the operation time T1 and the determination time T3 do not temporally overlap, but the operation time T1 and the determination time T3 may at least partially overlap.

Next, when the controller 20 determines that the pulse wave data does not satisfy the predetermined condition related to the pulse wave data (NO in step S42) as a result of the determination process in step S41, the controller 20 causes the physiological sensors 30 to stand by during the standby time T2 (step S43). Thereafter, the controller 20 obtains the physiological information by causing the physiological sensors 30 to operate again during the operation time T1 (step S40).

On the other hand, when the controller 20 determines that the pulse wave data satisfies the predetermined condition related to the pulse wave data (YES in step S42) as the result of the determination process in step S41, the controller 20 causes the physiological sensors 30 to stand by during the standby time T2 (step S44). Thereafter, the controller 20 obtains the physiological information by causing the physiological sensors 30 to operate again during an operation time T4 longer than the operation time T1 (T4>T1) (step S45).

As described above, due to the intermittent operation of the physiological sensors 30 according to the second embodiment, when the predetermined condition related to the pulse wave data is not satisfied, the physiological sensors 30 operate again during the operation time T1 after standing by. On the other hand, when the predetermined condition related to the pulse wave data is satisfied, the physiological sensors 30 operate again during the operation time T4 longer than the operation time T1 after standing by.

In the example of the time chart illustrated in FIG. 10, after the physiological sensors operate during the operation time T1, the determination result of the first step S42 is YES, and thus the physiological sensors 30 operate again during the operation time T4. Thereafter, the determination result of the next step S42 is NO, and thus the physiological sensors 30 operate again during the operation time T1. In the time chart illustrated in FIG. 10, the operation time T4 and the determination time T3 do not temporally overlap, but the operation time T4 and the determination time T3 may at least partially overlap.

First Specific Example of Determination Process Related to Pulse Wave Data

Next, a first specific example of the determination process related to the pulse wave data in step S41 will be specifically described below. In this regard, in step S41, the controller may determine that the patient P is not in a resting state by comparing the number of pulse waves included in the pulse wave data for 1 minute (that is, the pulse rate) with a predetermined threshold value. For example, when the pulse rate is larger than the predetermined threshold value, it may be determined that the patient P is not in the resting state. That is, it may be determined that the pulse wave data satisfies the predetermined condition. On the other hand, when the pulse rate is equal to or less than the predetermined threshold value, it may be determined that the patient P is in the resting state. That is, it may be determined that the pulse wave data does not satisfy the predetermined condition. When the patient P is not in the resting state (YES in step S42), it is estimated that the measurement accuracy of the physiological information decreases due to the motions of the patient, and thus the controller 20 increases the operation time of the physiological sensors 30 (T1 to T4). As described above, in the case where it is estimated that the measurement accuracy of the physiological information decreases, it is possible to compensate for the decrease in the measurement accuracy of the physiological information by increasing the operation time (the measurement time) of the physiological information.

Second Specific Example of Determination Process Related to Pulse Wave Data

Further, in step S41, the controller 20 may calculate a difference between the pulse rate included in the pulse wave data for 1 minute and a previously measured pulse rate (hereinafter referred to as a reference pulse rate), and then compare the difference with a predetermined threshold value so as to determine that the patient P is not in the resting state. For example, when the difference between the pulse rate and the reference pulse rate is larger than the predetermined threshold value, it may be determined that the patient P is not in the resting state. That is, it may be determined that the pulse wave data satisfies the predetermined condition. On the other hand, when the difference is equal to or less than the predetermined threshold value, it may be determined that the patient P is in the resting state. That is, it may be determined that the pulse wave data does not satisfy the predetermined condition. The reference pulse rate may be a representative value such as an average value or a median value of the previously measured pulse rate.

In the processes of step S41 described in the first specific example and the second specific example, a pulse amplitude index (PI), the transcutaneous arterial oxygen saturation (SpO2), the respiration rate, a signal quality index (SQI), and the like may be used as the parameters related to the pulse wave data.

Third Specific Example of Determination Process Related to Pulse Wave Data

When a difference between the pulse amplitude index and a predetermined threshold value is large (that is, when the pulse amplitude index is extremely low), the controller 20 may determine that the patient P is not in the resting state. On the other hand, when the difference between the pulse amplitude index and the predetermined threshold value is small, the controller 20 may determine that the patient P is in the resting state. Further, when the pulse amplitude index is smaller than the predetermined threshold value, the controller 20 may determine that the patient P is not in the resting state. On the other hand, when the pulse amplitude index is equal to or larger than the predetermined threshold value, the controller 20 may determine that the patient P is in the resting state. In addition, when a difference between the pulse amplitude index and a previously measured pulse amplitude index is larger than a predetermined threshold value, the controller 20 may determine that the patient P is not in the resting state. On the other hand, when the difference between the pulse amplitude index and the previously measured pulse amplitude index is equal to or less than the predetermined threshold value, the controller 20 may determine that the patient P is in the resting state.

Fourth Specific Example of Determination Process Related to Pulse Wave Data

When a difference between the SpO2 and a predetermined threshold value is large (that is, when the SpO2 is extremely low), the controller 20 may determine that the patient P is not in the resting state. On the other hand, when the difference between the SpO2 and the predetermined threshold value is small, the controller 20 may determine that the patient P is in the resting state. Further, when the SpO2 is smaller than the predetermined threshold value, the controller 20 may determine that the patient P is not in the resting state. On the other hand, when the SpO2 is equal to or larger than the predetermined threshold value, the controller 20 may determine that the patient P is in the resting state. In addition, when a difference between the SpO2 and a previously measured SpO2 is larger than a predetermined threshold value, the controller 20 may determine that the patient P is not in the resting state. On the other hand, when the difference between the SpO2 and the previously measured SpO2 is equal to or less than the predetermined threshold value, the controller 20 may determine that the patient P is in the resting state.

Fifth Specific Example of Determination Process Related to Pulse Wave Data

The controller 20 may calculate the respiration rate of the patient P based on the pulse wave data, and then determine whether the patient P is in the resting state by comparing the calculated respiration rate with a predetermined threshold value. For example, when the respiration rate is larger than the predetermined threshold value, the controller 20 may determine that the patient P is not in the resting state. On the other hand, when the respiration rate is larger than 0 and equal to or less than the predetermined threshold value, the controller may determine that the patient P is in the resting state. The predetermined threshold value related to the respiration rate may be determined based on a previously measured respiration rate.

Sixth Specific Example of Determination Process Related to Pulse Wave Data

The controller 20 may calculate the signal quality index (SQI) based on the pulse wave data, and then determine whether the patient P is in the resting state by comparing the calculated SQI with a predetermined threshold value. For example, when the SQI is smaller than the predetermined threshold value, the controller 20 may determine that the patient P is not in the resting state. On the other hand, when the SQI is equal to or larger than the predetermined threshold value, the controller 20 may determine that the patient P is in the resting state. The predetermined threshold value related to the SQI may be determined based on a previously measured SQI.

Further, in the second embodiment, it is determined that the patient P is not in the resting state by using the pulse wave data, but the physiological information used for the determination process is not limited to the pulse wave data. In this regard, the controller 20 may determine that the patient P is not in the resting state by using the electrocardiogram data. More specifically, the controller 20 may execute the determination process related to the electrocardiogram data in step S41.

First Specific Example of Determination Process Related to Electrocardiogram Data

Next, a first specific example of the determination process related to the electrocardiogram data will be specifically described below. In this regard, in step S41, the controller 20 may determine that the patient P is not in the resting state by comparing the number of heart rate waveforms included in electrocardiogram data for 1 minute (that is, the heart rate) with a predetermined threshold value. For example, when the heart rate is larger than the predetermined threshold value, it may be determined that the patient P is not in the resting state. That is, it may be determined that the electrocardiogram data satisfies the predetermined condition. On the other hand, when the heart rate is equal to or less than the predetermined threshold value, it may be determined that the patient P is in the resting state. That is, it may be determined that the electrocardiogram data does not satisfy the predetermined condition. When the patient P is not in the resting state (YES in step S42), it is estimated that the measurement accuracy of the physiological information decreases due to the motions of the patient, and thus the controller 20 increases the operation time of the physiological sensors 30 (T1 to T4). As described above, in the case where it is estimated that the measurement accuracy of the physiological information decreases, it is possible to compensate for the decrease in the measurement accuracy of the physiological information by increasing the operation time (the measurement time) of the physiological information.

Second Specific Example of Determination Process Related to Electrocardiogram Data

Further, in step S41, the controller 20 may calculate a difference between the heart rate included in the electrocardiogram data for 1 minute and a previously measured heart rate (hereinafter referred to as a reference heart rate), and then compare the difference with a predetermined threshold value so as to determine that the patient P is not in the resting state. For example, when the difference between the heart rate and the reference heart rate is larger than the predetermined threshold value, it may be determined that the patient P is not in the resting state. That is, it may be determined that the electrocardiogram data satisfies the predetermined condition. On the other hand, when the difference is equal to or less than the predetermined threshold value, it may be determined that the patient P is in the resting state. That is, it may be determined that the electrocardiogram data does not satisfy the predetermined condition. The reference heart rate may be a representative value such as an average value or a median value of the previously measured heart rate.

Third Specific Example of Determination Process Related to Electrocardiogram Data

Further, in step S41, the controller 20 may calculate the respiration rate of the patient P based on the electrocardiogram data for 1 minute, then calculate a difference between the calculated respiration rate and a previously measured respiration rate (hereinafter referred to as a reference respiration rate), and then compare the difference with a predetermined threshold value so as to determine that the patient P is not in the resting state. For example, when the difference between the respiration rate and the reference respiration rate is larger than the predetermined threshold value, it may be determined that the patient P is not in the resting state. That is, it may be determined that the electrocardiogram data satisfies the predetermined condition. On the other hand, when the difference is equal to or less than the predetermined threshold value, it may be determined that the patient P is in the resting state. That is, it may be determined that the electrocardiogram data does not satisfy the predetermined condition. The reference respiration rate may be a representative value such as an average value or a median value of the previously measured respiration rate.

Fourth Specific Example of Determination Process Related to Electrocardiogram Data

In step S41, the controller 20 may determine whether arrhythmia exists in the heart rate waveforms included in the electrocardiogram data, and then determine whether the patient P is in the resting state according to the presence or absence of the arrhythmia. Specifically, the controller 20 may determine that the patient P is not in the resting state when the arrhythmia exists in the electrocardiogram data. On the other hand, the controller 20 may determine that the patient P is in the resting state when no arrhythmia exists in the electrocardiogram data.

### Third Embodiment

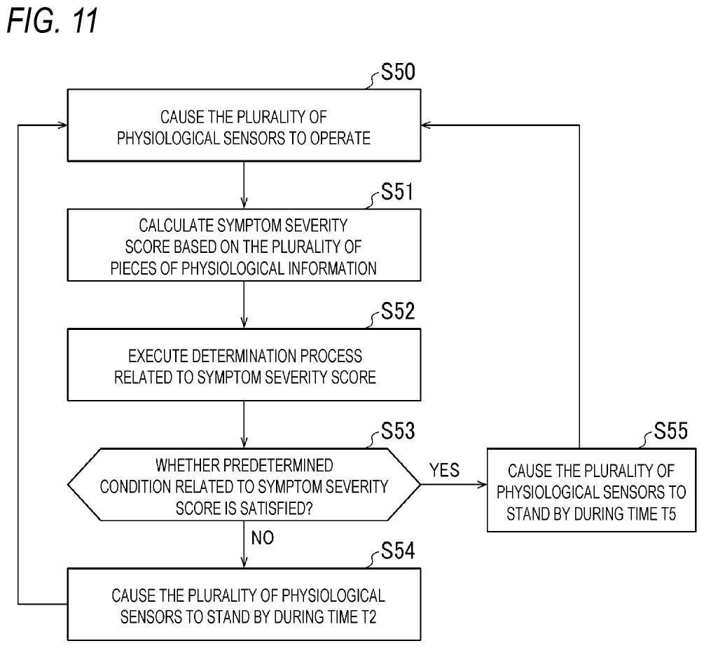

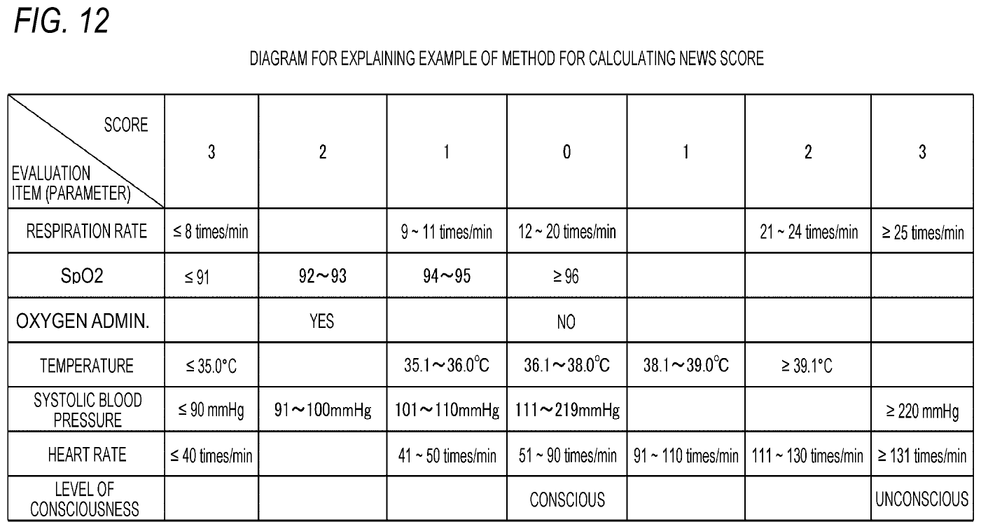

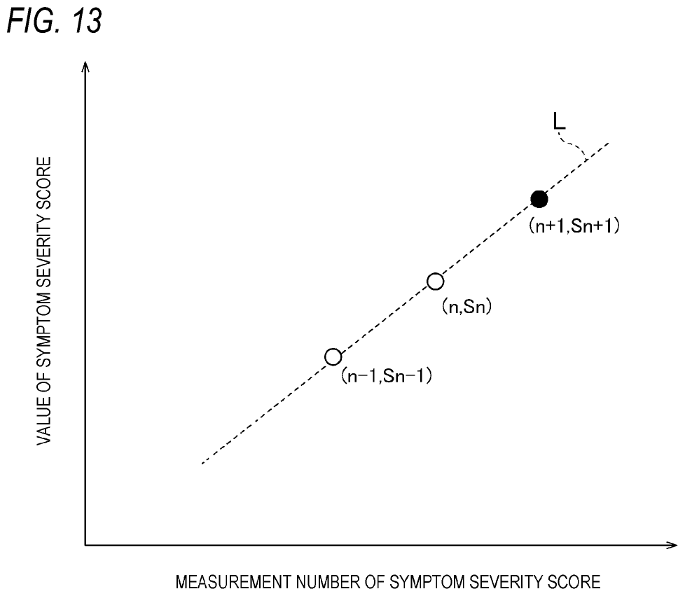

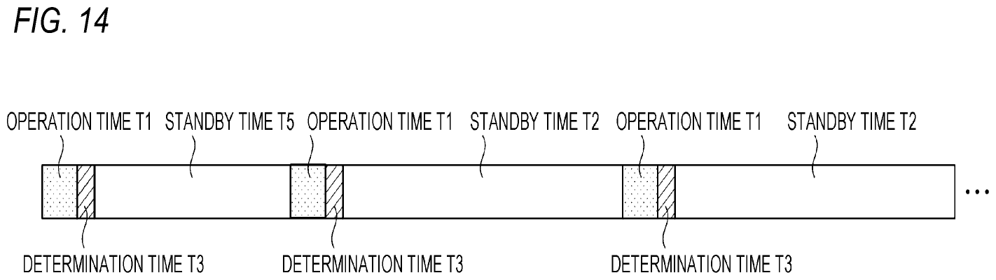

Next, an intermittent operation of the physiological sensors 30 according to a third embodiment will be described below with reference to FIGS. 11 and 14. FIG. 11 is a flow chart for explaining the intermittent operation of the physiological sensors 30 according to the third embodiment. FIG. 12 is a table for explaining an example of a method for calculating a NEWS score (an example of a symptom severity score). FIG. 13 illustrates an example of a temporal transition of the calculated symptom severity score. FIG. 14 is a time chart illustrating the example of the intermittent operation of the physiological sensors 30 according to the third embodiment.

As illustrated in FIG. 11, in step S50, the controller 20 causes the physiological sensors 30 (the electrocardiogram sensor 31, the pulse wave sensor 32, the body motion sensor 33, the temperature sensor 34, and the skin potential sensor 35) to operate during the operation time T1 (see FIG. 14) so as to obtain the plurality of pieces of physiological information (the electrocardiogram data, the heart rate data, the pulse wave data, the pulse rate data, the SpO2 data, the body motion data, the temperature data, and the skin potential data). In step S51, the controller 20 calculates the symptom severity score of the patient P based on the plurality of pieces of physiological information.

For example, the controller 20 may calculate the NEWS score as the example of the symptom severity score of the patient P. As illustrated in FIG. 12, the controller 20 can calculate the NEWS score based on a plurality of pieces of vital data, the presence or absence of oxygen administration, and the presence or absence of consciousness of the patient. Specifically, the controller 20 calculates respective sub-scores of the plurality of pieces of vital data in accordance with respective comparisons between the plurality of pieces of vital data and reference ranges set for the plurality of pieces of vital data. For example, as illustrated in FIG. 12, when the respiration rate (RR) is 12 to 20 times/min, a sub-score related to the respiration rate is 0. When the respiration rate is 9 to 11 times/min, the sub-score related to the respiration rate is 1. When the respiration rate is 21 to 24 times/min, the sub-score related to the respiration rate is 2. When the respiration rate is 8 times/min or less or 25 times/min or more, the sub-score related to the respiration rate is 3. Further, the controller 20 may calculate a sub-score related to the oxygen administration and a sub-score related to the consciousness of the patient. For example, when the oxygen administration is performed, the sub-score related to the oxygen administration is 2. On the other hand, when the oxygen administration is not performed, the sub-score related to the oxygen administration is 0. Further, when the patient is unconscious, the sub-score related to the consciousness of the patient is 3. On the other hand, when the patient is conscious, the sub-score related to the consciousness of the patient is 0. In this regard, the processing apparatus 2 may periodically obtain, from the patient database 6 via the in-hospital network 3, information on the sub-score related to the oxygen administration and the sub-score related to the consciousness of the patient. Further, as described above, the processing apparatus 2 may obtain information on the respiration rate of the patient based on the electrocardiogram data.

In this manner, the controller 20 can calculate the NEWS score of the patient by summing the calculated sub-scores. Next, in step S52, the controller 20 executes a determination process related to the symptom severity score (in this example, the NEWS score) during the determination time T3. A specific example of the determination process in step S52 will be described later. In the time chart illustrated in FIG. 14, the operation time T1 and the determination time T3 do not temporally overlap, but the operation time T1 and the determination time T3 may at least partially overlap.

Further, in the present embodiment, the symptom severity score is not limited to the NEWS score. For example, an SOFA, a qSOFA, an APACHE2, a BSAS, a NIHSS, an NEWS2, a MEWS, or the like may be adopted as other examples of the symptom severity score. In the present embodiment, the respiration rate, the transcutaneous arterial oxygen saturation, the temperature, a systolic blood pressure, and the heart rate are used as the vital data of the patient in order to calculate the NEWS score, but types of the vital data of the patient to be used may be changed according to the type of the symptom severity score to be adopted.

Next, when the controller 20 determines that the symptom severity score does not satisfy the predetermined condition related to the symptom severity score (NO in step S53) as a result of the determination process in step S52, the controller 20 causes the physiological sensors 30 to stand by during the standby time T2 (step S54). Thereafter, the controller 20 obtains the physiological information by causing the physiological sensors 30 to operate again during the operation time T1 (step S50).

On the other hand, when the controller 20 determines that the symptom severity score satisfies the predetermined condition related to the symptom severity score (YES in step S53) as the result of the determination process in step S52, the controller 20 causes the physiological sensors 30 to stand by during a standby time T5 shorter than the standby time T2 (step S55). Thereafter, the controller 20 obtains the physiological information by causing the physiological sensors 30 to operate again during the operation time T1 (step S50).

As described above, due to the intermittent operation of the physiological sensors 30 according to the third embodiment, when the predetermined condition related to the symptom severity score is not satisfied, the physiological sensors 30 operate again during the operation time T1 after standing by during the standby time T2. On the other hand, when the predetermined condition related to the symptom severity score is satisfied, the physiological sensors 30 operate again during the operation time T4 after standing by during the standby time T5 shorter than the standby time T2.

In the example of the time chart illustrated in FIG. 14, after the physiological sensors operate during the operation time T1, the determination result of the first step S53 is YES, and thus the physiological sensors 30 operate again after standing by during the standby time T5. Thereafter, since the determination result of the next step S53 is NO, the physiological sensors 30 operate again after standing by during the standby time T2.

First Specific Example of Determination Process Related to Symptom Severity Score

Next, a first specific example of the determination process related to the symptom severity score in step S52 will be specifically described below. In this regard, in step S52, the controller 20 may compare the calculated symptom severity score with the predetermined threshold value to determine whether the symptoms of the patient P become serious. For example, when the symptom severity score is larger than the predetermined threshold value, it may be determined that the symptoms of the patient P become serious. That is, it may be determined that the symptom severity score satisfies the predetermined condition. On the other hand, when the calculated symptom severity score is equal to or less than the predetermined threshold value, it may be determined that the symptoms of the patient P do not become serious. That is, it may be determined that the symptom severity score does not satisfy the predetermined condition. When the symptoms of the patient P become serious (YES in step S53), the controller increases the measurement frequency of the physiological information by shortening the standby time from T2 to T5. In this manner, by increasing the measurement frequency of the physiological information of the patient P, it is possible to obtain more physiological information on the seriously ill patient from the physiological sensors 30. Accordingly, it is possible to optimize the intermittent operation of the physiological sensors 30 according to conditions of the patient P.

Second Specific Example of Determination Process Related to Symptom Severity Score

Next, a second specific example of the determination process related to the symptom severity score in step S52 will be described below. In this regard, in step S52, the controller 20 estimates a symptom severity score to be calculated next based on the currently calculated symptom severity score and a previously calculated symptom severity score. For example, as illustrated in FIG. 13, when a symptom severity score Sn calculated at the n-th time (n is a natural number equal to or larger than 2) is the currently calculated symptom severity score, the previously calculated symptom severity score is S(n−1). A vertical axis of a graph illustrated in FIG. 13 is a value of the symptom severity score. A horizontal axis of the graph indicates a measurement number of the symptom severity score. That is, the horizontal axis indicates a time. A time interval between a time at which the n-th symptom severity score Sn is calculated and a time at which the (n−1)th symptom severity score S(n−1) is calculated may correspond to the total time of the operation time T1+the determination time T3+the standby time T5. The controller 20 calculates a regression line L indicating a temporal change in the symptom severity score based on two symptom severity scores including the currently calculated symptom severity score Sn and the previously calculated symptom severity score S(n−1). Thereafter, the controller 20 estimates a symptom severity score S(n+1) to be calculated next by using the regression line L.

Next, the controller 20 may compare the next symptom severity score S(n+1) estimated by using the regression line L with the predetermined threshold value to determine whether the symptoms of the patient P become serious from the current time. For example, when the next symptom severity score S(n+1) is larger than the predetermined threshold value, it may be determined that the symptoms of the patient P become serious from the current time. That is, it may be determined that the symptom severity score satisfies the predetermined condition. On the other hand, when the next symptom severity score S(n+1) is equal to or less than the predetermined threshold value, it may be determined that the symptoms of the patient P do not become serious from the current time. That is, it may be determined that the symptom severity score does not satisfy the predetermined condition. When the symptoms of the patient P become serious from the current time (YES in step S53), the controller 20 increases the measurement frequency of the physiological information by shortening the standby time from T2 to T5. In this manner, by increasing the measurement frequency of the physiological information of the patient P, it is possible to obtain more physiological information on the seriously ill patient from the physiological sensors 30. Accordingly, it is possible to optimize the intermittent operation of the physiological sensors 30 according to the conditions of the patient P. As the second specific example, the next symptom severity score is estimated based on the regression line L indicating the temporal change in the symptom severity score, but the next symptom severity score may be estimated based on a Kalman filter, a particle filter, a state space model, a statistical and time-sequential model, data assimilation, recurrent neural network (RNN), long short term memory (LSTM), or the like as other analysis methods.

### Fourth Embodiment

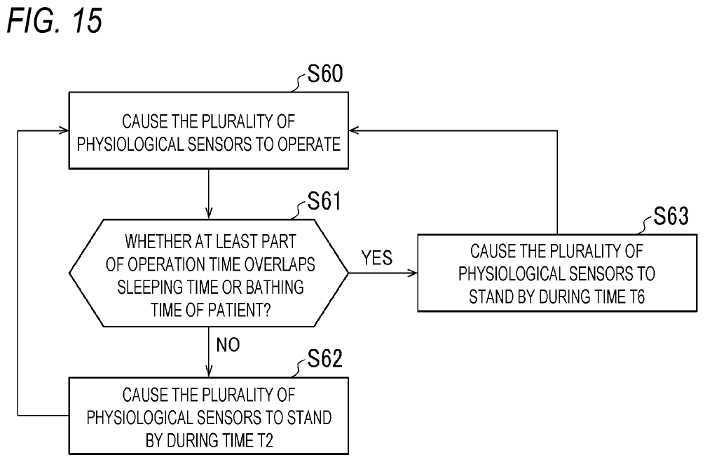

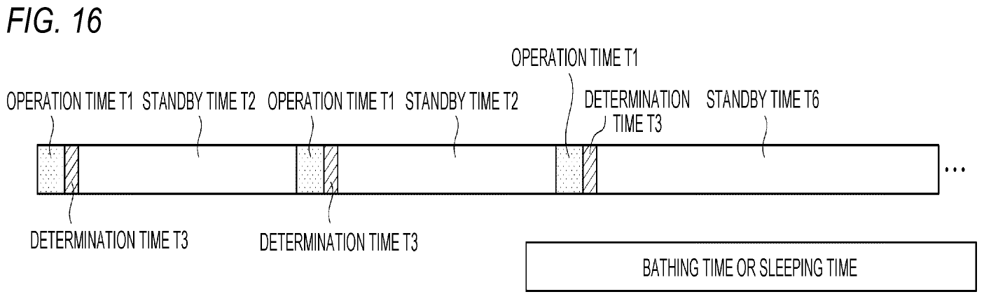

Next, an intermittent operation of the physiological sensors 30 according to a fourth embodiment will be described below with reference to FIGS. 15 and 16. FIG. 15 is a flow chart for explaining the intermittent operation of the physiological sensors 30 according to the fourth embodiment. FIG. 16 is a time chart illustrating an example of the intermittent operation of the physiological sensors 30 according to the fourth embodiment.

As illustrated in FIG. 15, in step S60, the controller 20 causes the physiological sensors 30 (the electrocardiogram sensor 31, the pulse wave sensor 32, the body motion sensor 33, the temperature sensor 34, and the skin potential sensor 35) to operate during the operation time T1 (see FIG. 16) so as to obtain the plurality of pieces of physiological information (the electrocardiogram data, the heart rate data, the pulse wave data, the pulse rate data, the SpO2 data, the body motion data, the temperature data, and the skin potential data). In step S61, the controller 20 determines whether at least a part of the operation time T1 overlaps a sleeping time or a bathing time (an example of a predetermined living time period) of the patient P. The processing apparatus 2 may periodically receive, from the server 4 via the in-hospital network 3, information on the bathing time and the sleeping time of the patient P.

Next, when the controller 20 determines that at least a part of the operation time T1 does not overlap the sleeping time or the bathing time of the patient P (NO in step S61) as a result of a determination process in step S61, the controller 20 causes the physiological sensors to stand by during the standby time T2 (step S62). Thereafter, the controller 20 obtains the physiological information by causing the physiological sensors 30 to operate again (step S60).

On the other hand, when the controller 20 determines that at least a part of the operation time T1 overlaps the sleeping time or the bathing time of the patient P (YES in step S61) as the result of the determination process in step S61, the controller 20 causes the physiological sensors 30 to stand by during a standby time T6 longer than the standby time T2 (step S63). Thereafter, the controller 20 obtains the physiological information by causing the physiological sensors 30 to operate again (step S60).

As described above, due to the intermittent operation of the physiological sensors 30 according to the fourth embodiment, when at least a part of the operation time T1 does not overlap the sleeping time or the bathing time of the patient P, the physiological sensors 30 operate again during the operation time T1 after standing by during the standby time T2. On the other hand, when at least a part of the operation time T1 overlaps the sleeping time or the bathing time of the patient P, the physiological sensors 30 operate again during the operation time T1 after standing by during the standby time T6 (T6>T2).

In the example of the time chart illustrated in FIG. 16, after the physiological sensors stand by twice during the standby time T2, the determination result of step S61 is YES, and thus the physiological sensors 30 stand by during the standby time T6 longer than the standby time T2. When the patient P takes a bath (YES in step S61), it is estimated that the physiological information on the patient P cannot be accurately measured, and thus the controller 20 increases the standby time of the physiological sensors 30 so as to decrease the measurement frequency of the physiological information. Therefore, it is possible to further reduce the power consumption of the battery by the processing apparatus 2. On the other hand, when the patient P sleeps (YES in step S61), it is estimated that the physiological information on the patient P is stable, and thus the controller 20 increases the standby time of the physiological sensors 30 so as to decrease the measurement frequency of the physiological information. Therefore, it is possible to further reduce the power consumption of the battery 26 by the processing apparatus 2.

In the description of the present embodiment, when the operation time of the physiological sensors 30 at least partially overlaps the bathing time or the sleeping time, the controller 20 increases the standby time of the physiological sensors 30, but the present embodiment is not limited thereto. For example, when the operation time of the physiological sensors 30 at least partially overlaps an examination time of the patient P, the controller 20 may increase the standby time of the physiological sensors 30. Since there is a high possibility that the processing apparatus 2 is temporarily detached from the patient P during the examination of the patient P, it is estimated that the processing apparatus 2 cannot accurately measure the physiological information on the patient P. Therefore, the controller 20 increases the standby time of the physiological sensors 30, and thus the power consumption of the battery 26 can be further reduced.

Due to the intermittent operations according to the first embodiment to the fourth embodiment, an operation mode of the plurality of physiological sensors 30 that intermittently operate (for example, remeasurement of the physiological information, extension or reduction of the operation time or the standby time) is changed in accordance with a condition related to at least a part of the plurality of pieces of physiological information or a condition related to at least a part of the plurality of physiological sensors 30. As described above, for example, even in the situation where the physiological information of the physiological sensors 30 cannot be temporarily accurately measured due to movements such as motions (for example, walking) of the patient P, it is possible to suitably prevent the decrease in the measurement accuracy of the physiological information measured by the physiological sensors 30 while restraining the power consumption of the processing apparatus 2. Further, when the symptoms of the patient P are serious, it is possible to obtain more physiological information on the seriously ill patient from the physiological sensors 30 while restraining the power consumption of the processing apparatus 2. Accordingly, it is possible to provide the processing apparatus 2 that is capable of optimizing the intermittent operation of the physiological sensors 30 according to the conditions of the patient P.

Alarm Presentation Process

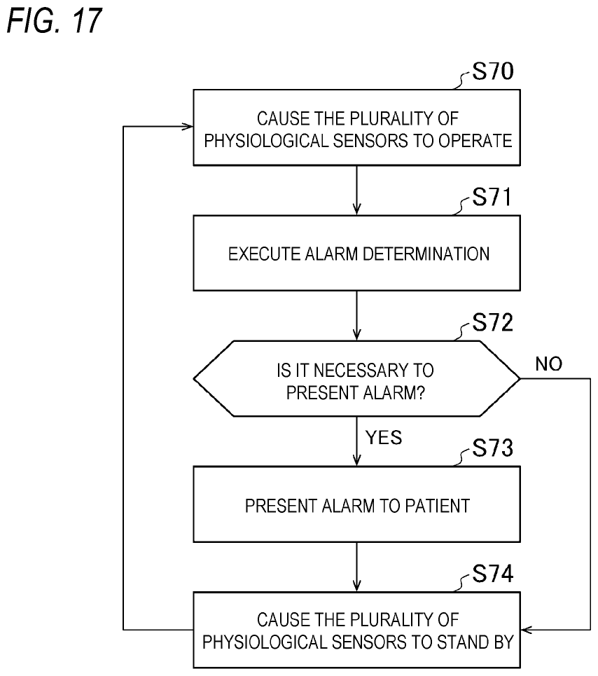

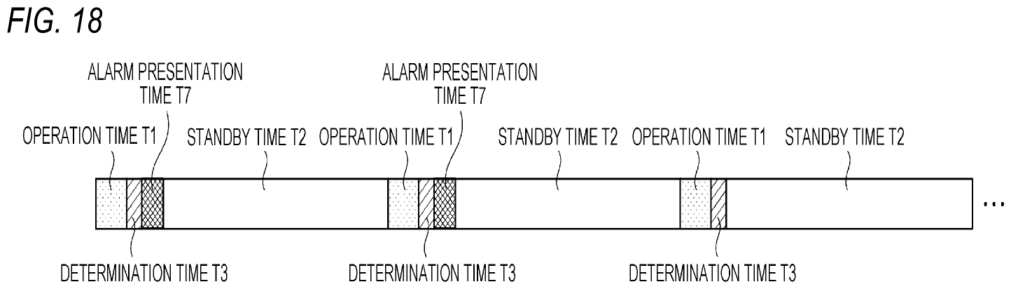

Next, a process of presenting an alarm (a warning) to the patient P will be described below with reference to FIGS. 17 and 18. FIG. 17 is a flow chart for explaining the process of presenting an alarm to the patient P. FIG. 18 is a time chart illustrating an example of the intermittent operation of the physiological sensors, which includes an alarm presentation time T7.

As illustrated in FIG. 17, in step S70, the controller 20 causes the physiological sensors 30 (the electrocardiogram sensor 31, the pulse wave sensor 32, the body motion sensor 33, the temperature sensor 34, and the skin potential sensor 35) to operate during the operation time T1 (see FIG. 18) so as to obtain the plurality of pieces of physiological information (the electrocardiogram data, the heart rate data, the pulse wave data, the pulse rate data, the SpO2 data, the body motion data, the temperature data, and the skin potential data). In step S71, the controller 20 determines whether an alarm should be presented to the patient P.

Specifically, the controller 20 may determine whether the skin potential of the patient P is equal to or less than a predetermined threshold value based on the skin potential data of the patient P. When the skin potential of the patient P is equal to or less than the predetermined threshold value, the controller 20 may determine that the skin potential sensor 35 is not in contact with the patient P. That is, the controller 20 determines that the processing apparatus 2 is not in contact with the patient P, and then determines that the alarm should be presented to the patient P. On the other hand, when the skin potential of the patient P is larger than the predetermined threshold value, the controller 20 may determine that the skin potential sensor is in contact with the patient P. That is, the controller 20 determines that the processing apparatus 2 is in contact with the patient P, and then determines that it is not necessary to present the alarm to the patient P.

Further, the controller 20 may determine whether a skin resistance of the patient P is equal to or larger than a predetermined threshold value. When the skin resistance of the patient P is equal to or larger than the predetermined threshold value, the controller 20 may determine that the skin potential sensor 35 is not in contact with the patient P. That is, the controller 20 determines that the processing apparatus 2 is not in contact with the patient P, and then determines that the alarm should be presented to the patient P. On the other hand, when the skin resistance of the patient P is smaller than the predetermined threshold value, the controller 20 may determine that the skin potential sensor 35 is in contact with the patient P. That is, the controller 20 determines that the processing apparatus 2 is in contact with the patient P, and then determines that it is not necessary to present the alarm to the patient P.

Further, the controller 20 may determine whether the temperature of the patient P is equal to or less than a predetermined threshold value based on the temperature data of the patient P. When the temperature of the patient P is equal to or less than the predetermined threshold value, the controller 20 may determine that the temperature sensor 34 is not in contact with the patient P. That is, the controller 20 determines that the processing apparatus 2 is not in contact with the patient P, and then determines that the alarm should be presented to the patient P. On the other hand, when the temperature of the patient P is larger than the predetermined threshold value, the controller 20 may determine that the temperature sensor 34 is in contact with the patient P. That is, the controller 20 determines that the processing apparatus 2 is in contact with the patient P, and then determines that it is not necessary to present the alarm to the patient P.

In addition, the controller 20 may determine whether the processing apparatus 2 is in contact with the patient P based on the pulse wave data or the electrocardiogram data of the patient P. For example, when normal pulse wave data and/or electrocardiogram data cannot be obtained, the controller 20 may determine that the processing apparatus 2 is not in contact the patient P and then determine that the alarm should be presented to the patient P. On the other hand, when the normal pulse wave data and/or electrocardiogram data can be obtained, the controller 20 may determine that the processing apparatus 2 is in contact with the patient P and then determine that it is not necessary to present the alarm to the patient P.

When the controller 20 determines that it is necessary to present the alarm to the patient P (YES in step S72), the controller 20 presents the alarm to the patient P visually, audibly, and/or tactically via the notification unit 23 (step S73). Thereafter, the controller 20 causes the physiological sensors 30 to stand by in step S74 and then causes the physiological sensors 30 to operate again (step S70). On the other hand, when the controller 20 determines that it is not necessary to present the alarm to the patient P (NO in step S72), the controller 20 causes the physiological sensors 30 to operate again (step S70) after causing the physiological sensors 30 to stand by in step S74.

In the example of the time chart illustrated in FIG. 18, since the determination result of the first step S72 is YES, the processing apparatus 2 presents the alarm to the patient P during the alarm presentation time T7. Since the determination result of the second step S72 is also YES, the processing apparatus 2 presents the alarm to the patient P during the alarm presentation time T7. Since the determination result of the third step S72 is NO, the processing apparatus 2 does not present the alarm to the patient P. The alarm presentation time T7 may partially overlap the standby time T2.

According to the present embodiment, when the processing apparatus 2 is not in contact with the skin of the patient P, the alarm is presented to the patient P visually, audibly, and/or tactically. In this manner, the patient P can immediately recognize that the physiological information on the patient is not accurately measured by the processing apparatus 2 by the alarm. As described above, it is possible to suitably prevent a situation where the physiological information on the patient P cannot be measured for a long period by the physiological sensors 30.

In the description of the present embodiment, the alarm is presented to the patient P via the notification unit 23, but the present embodiment is not limited thereto. For example, a message indicating that the processing apparatus 2 is not correctly attached to the patient P may be transmitted to a mobile terminal (not illustrated) such as a smartphone carried by the patient P via the in-hospital network 3 or the Internet.

Further, in order to implement the series of processes executed by the processing apparatus 2 through software, the physiological information processing program may be incorporated in the storage device 21 or the ROM in advance. Alternatively, the physiological information processing program may be stored in a computer readable storage medium such as a magnetic disk (for example, HDD and a floppy disk), an optical disk (for example, CD-ROM, DVD-ROM, and Blu-ray (registered trademark) disk), a magneto optical disk (for example, MO), a flash memory (for example, a SD card, a USB memory, and SSD). In this case, the physiological information processing program stored in the storage medium may be incorporated in the storage device 21. After the physiological information processing program incorporated in the storage device 21 is loaded onto the RAM, the processor may execute the physiological information processing program loaded on the RAM.

Further, the physiological information processing program may be downloaded from a server on a communication network such as the Internet. In this case, the downloaded program may be incorporated into the storage device 21.

Although the embodiments of the presently disclosed subject matter have been described above, the technical scope of the presently disclosed subject matter should not be construed as being limited to the description of the present embodiments. The present embodiments are merely examples, and it is understood by those skilled in the art that various modifications of the embodiments are possible within the scope of the disclosed subject matters described in the claims. The technical scope of the presently disclosed subject matter should be determined based on the scope of the disclosed subject matters described in the claims and equivalents thereof.

In addition, in the present description, the intermittent operations of the physiological sensors 30 according to the first embodiment to the fourth embodiment have been described, and two or more intermittent operations among these intermittent operations may be combined. That is, the controller 20 may control the operation of the physiological sensors 30 to simultaneously execute at least two intermittent operations among the intermittent operations according to the first embodiment to the fourth embodiment. Further, the process of presenting an alarm to the patient P may be executed in each of the intermittent operations according to the first embodiment to the fourth embodiment.

In the intermittent operation according to each embodiment, the controller 20 causes the physiological sensors 30 to intermittently operate, but a part of the plurality of physiological sensors 30 may always operate. For example, the controller 20 may cause the physiological sensors 30 other than the body motion sensor 33 to intermittently operate according to conditions of the patient P while causing the body motion sensor 33 to always operate.

1. A physiological information processing apparatus, comprising:
one or more processors; and
one or more memories configured to store a computer readable instruction, wherein
when the computer readable instruction is executed by the processor, the physiological information processing apparatus is configured to:
cause a plurality of physiological sensors to intermittently operate such that an operation time and a standby time are alternately repeated,
obtain a plurality of pieces of physiological information on a patient from the plurality of physiological sensors during the operation time, and
change an operation mode of the plurality of physiological sensors that intermittently operate in accordance with a condition related to at least a part of the plurality of pieces of physiological information or a condition related to at least a part of the plurality of physiological sensors.

2. The physiological information processing apparatus according to claim 1, wherein
the physiological information processing apparatus is configured to:
determine whether first physiological information among the plurality of pieces of physiological information satisfies a predetermined condition associated with the first physiological information,
when the first physiological information does not satisfy the predetermined condition, cause the plurality of physiological sensors to stand by during the standby time and after the standby time finished, cause the plurality of physiological sensors to operate, and
when the first physiological information satisfies the predetermined condition, cause the plurality of physiological sensors to operate again without causing the plurality of physiological sensors to stand by.

3. The physiological information processing apparatus according to claim 2, wherein
the first physiological information includes electrocardiogram data, pulse wave data, or body motion data of the patient.

4. The physiological information processing apparatus according to claim 1, wherein
the physiological information processing apparatus is configured to:
determine whether first physiological information among the plurality of pieces of physiological information satisfies a predetermined condition associated with the first physiological information,
set the operation time to a first time when the first physiological information does not satisfy the predetermined condition, and
set the operation time to a second time longer than the first time when the first physiological information satisfies the predetermined condition.

5. The physiological information processing apparatus according to claim 4, wherein
the first physiological information includes pulse wave data or electrocardiogram data of the patient.

6. The physiological information processing apparatus according to claim 1, wherein
the physiological information processing apparatus is configured to:
calculate a symptom severity score indicating a symptom severity of the patient based on the plurality of pieces of physiological information,
determine whether the symptom severity score satisfies a predetermined condition associated with the symptom severity score,
set the standby time to a first time when the symptom severity score does not satisfy the predetermined condition, and
set the standby time to a second time shorter than the first time when the symptom severity score satisfies the predetermined condition.

7. The physiological information processing apparatus according to claim 6, wherein
the physiological information processing apparatus is configured to:
determine whether the symptom severity score is larger than a predetermined threshold value,
set the standby time to the first time when the symptom severity score is equal to or less than the predetermined threshold value, and
set the standby time to the second time when the symptom severity score is larger than the predetermined threshold value.

8. The physiological information processing apparatus according to claim 6, wherein
the physiological information processing apparatus is configured to:
estimate a symptom severity score to be calculated next based on a currently calculated symptom severity score and at least one previously calculated symptom severity score,
determine whether the estimated symptom severity score is larger than a predetermined threshold value,
set the standby time to the first time when the estimated symptom severity score is equal to or less than the predetermined threshold value, and
set the standby time to the second time when the estimated symptom severity score is larger than the predetermined threshold value.

9. The physiological information processing apparatus according to claim 1, wherein
the physiological information processing apparatus is configured to:
determine whether at least a part of the operation time overlaps a predetermined living time period of the patient,
set the standby time to a first time when at least a part of the operation time does not overlap the predetermined living time period, and
set the standby time to a second time longer than the first time when at least a part of the operation time overlaps the predetermined living time period.

10. The physiological information processing apparatus according to claim 1, wherein
the physiological information processing apparatus is configured to:
determine whether a first physiological sensor among the plurality of physiological sensors is in contact with the skin of the patient based on first physiological information obtained by the first physiological sensor, and
present an alarm to the patient when the first physiological sensor is not in contact with the skin of the patient.

11. A physiological information processing apparatus, comprising:
one or more processors; and
one or more memories configured to store a computer readable instruction, wherein
when the computer readable instruction is executed by the processor, the physiological information processing apparatus is configured to:
cause a plurality of physiological sensors to intermittently operate such that an operation time and a standby time are alternately repeated,
obtain a plurality of pieces of physiological information on a patient from the plurality of physiological sensors during the operation time,
determine whether a first physiological sensor among the plurality of physiological sensors is in contact with the skin of the patient based on first physiological information obtained by the first physiological sensor, and
present an alarm to the patient when the first physiological sensor is not in contact with the skin of the patient.

12. The physiological information processing apparatus according to claim 1, wherein
the physiological information processing apparatus is attached to the patient, and
the physiological information processing apparatus further comprises a wireless communication module configured to wirelessly communicate with an external device.

13. A physiological information processing method to be executed by a computer, comprising:
causing a plurality of physiological sensors to intermittently operate such that an operation time and a standby time are alternately repeated;
obtaining a plurality of pieces of physiological information on a patient from the plurality of physiological sensors during the operation time; and
changing an operation mode of the plurality of physiological sensors that intermittently operate in accordance with a condition related to at least a part of the plurality of pieces of physiological information or a condition related to at least a part of the plurality of physiological sensors.

14. The physiological information processing method according to claim 13, further comprising:
determining whether first physiological information among the plurality of pieces of physiological information satisfies a predetermined condition associated with the first physiological information, wherein
changing the operation mode of the plurality of physiological sensors includes
when the first physiological information does not satisfy the predetermined condition, causing the plurality of physiological sensors to stand by during the standby time and after the standby time finished, causing the plurality of physiological sensors to operate, and
when the first physiological information satisfies the predetermined condition, causing the plurality of physiological sensors to operate again without causing the plurality of physiological sensors to stand by.

15. The physiological information processing method according to claim 13, further comprising:
determining whether first physiological information among the plurality of pieces of physiological information satisfies a predetermined condition associated with the first physiological information, wherein
changing the operation mode of the plurality of physiological sensors includes
setting the operation time to a first time when the first physiological information does not satisfy the predetermined condition, and
setting the operation time to a second time longer than the first time when the first physiological information satisfies the predetermined condition.

16. The physiological information processing method according to claim 13, further comprising:
calculating a symptom severity score indicating a symptom severity of the patient based on the plurality of pieces of physiological information; and
determining whether the symptom severity score satisfies a predetermined condition associated with the symptom severity score, wherein
changing the operation mode of the plurality of physiological sensors includes
setting the standby time to a first time when the symptom severity score does not satisfy the predetermined condition, and
setting the standby time to a second time shorter than the first time when the symptom severity score satisfies the predetermined condition.

17. The physiological information processing method according to claim 13, further comprising:
determining whether at least a part of the operation time overlaps a predetermined living time period of the patient, wherein
changing the operation mode of the plurality of physiological sensors includes
setting the standby time to a first time when at least a part of the operation time does not overlap the predetermined living time period, and
setting the standby time to a second time longer than the first time when at least a part of the operation time overlaps the predetermined living time period.

18. A non-transitory computer readable storage medium storing a program for causing a computer to execute a physiological information processing method according to claim 13.
